(19) United States
(12) Patent Application Publication
(10) Pub. No.: US 2021/0000809 A1
(43) Pub. Date: Jan. 7, 2021

(54) PROCESS FOR THE PREPARATION OF ABAMETAPIR AND ITS PHARMACEUTICALLY ACCEPTABLE SALTS

(71) Applicant: Mylan Laboratories Limited, Hyderabad (IN)

(72) Inventors: Sureshbabu JAYACHANDRA, Hyderabad (IN); Madhuresh Sethi, Hyderabad (IN); Vipin Kumar KAUSHIK, Hyderabad (IN); Vijaya Krishna Ravi, Hyderabad (IN); Saiprasad Kottalla, Hyderabad (IN)

(73) Assignee: Mylan Laboratories Limited, Hyderabad (IN)

(21) Appl. No.: 16/980,162

(22) PCT Filed: Mar. 8, 2019
(86) PCT No.: PCT/IB2019/051887

§ 371 Date: Sep. 11, 2020

(30) Foreign Application Priority Data
Mar. 13, 2018 (IN) 201841009214

(51) Int. Cl. A61K 31/444

(52) U.S. Cl. CPC A61K 31/444; C07B 2200/13

(57) ABSTRACT

The present invention relates to process for the preparation of Abametapir. The present invention further relates to Abametapir salts and their preparation thereof.

This application claims the benefit of Indian Provisional Patent Application IN201841009214, filed on 13 Mar. 2018, hereby incorporated by reference in its entirety.

### FIELD OF THE INVENTION

The present invention relates to process for the preparation of Abametapir. The present invention further relates to Abametapir salts and their preparation thereof.

### BACKGROUND OF THE INVENTION

Abametapir is chemically known as 5-methyl-2-(5-methylpyridin-2-yl)pyridine having the structure shown in formula-I.

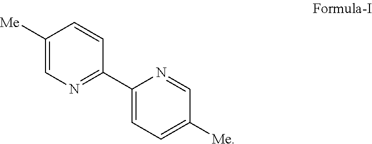 [SMILES: Cc1ccc(-c2ccc(C)cn2)nc1]

Abametapir is a Metalloprotease inhibitor and chelating agent, targeting proteases essential to insect hatching and survival. It affects multiple proteases essential to insect hatching and survival by chelating heavy metal ions, targeting all stages of the insect life cycle; it is therefore expected to be effective as a single application. The product is in phase III clinical development in the US.

Japanese patent JP1577703 first discloses a process for the preparation of Abametapir.

Tetrahedron Letters Vol. 39 Year 1998 Pg 2559 discloses process for the preparation of Abametapir comprising a mixture of 2-Bromo-5-Methyl pyridine, Pd(OAc)₂/nBu₄NBr DMF/H₂0, isopropanol and K₂CO₃ it gives final compound.

The journal of Organic Letters Vol. 02 Year 2000 Pg 3373-3376 discloses a process for the preparation of different methyl-substituted 2,2-bipyridines such as monomethyl-substituted, dimethyl-substituted. This process comprising a mixture of 2-tributylstannyl-picoline or 2-tributylstannyl-pyridine, 2-bromo-picoline or 2-bromo-pyridine, and triphenylphosphine-palladium(0) in 65 mL of toluene was refluxed under nitrogen for 48 h. The resulting brown mixture was evaporated in vacuum, and the dark, muddy liquid was dissolved in dichloromethane. The organic phase was washed with aqueous HCl. To remove the product from solution the combined aqueous layers were transferred dropwise in aqueous ammonia (10%) under cooling. The resulting oil was extracted with dichloromethane. The organic phases were washed with ammonia and water, and the solvent was removed. The resulting suspension was purified by column chromatography.

J. Hassan et al.: C. R. Acad. Sci. Paris, Se'rie IIc, Chimie: Chemistry 3 (2000) 517-521 discloses process for the preparation of Abametapir comprising a mixture of base, palladium acetate, tetra-n-butylammonium bromide and 2-Bromo-5-Methyl pyridine in a solvent or a mixture of DMF:H₂O was stirred under nitrogen atmosphere for a few minutes at 105° C. in the case of toluene as solvent or at 115° C. in the case of DMF. Isopropanol was added. After cooling to room temperature, water and ether were added. The organic phase was washed with water and dried over MgSO4. The solvent was evaporated under vacuum. The biphenyl product was purified by preparative thin layer chromatography or recrystallization.

Tetrahedron Letters V.44 Year 2003 Pg 2935 discloses process for the preparation of Abametapir comprising a mixture of 2-Bromo-5-Methyl pyridine, PdC₂(PPh₃)₂, DMF, isopropanol and K₃PO₄ it gives final compound.

The present inventors prepared Abametapir by an efficient, economical and industrially feasible process with good yields.

### OBJECT AND SUMMARY OF THE INVENTION

The main aspect of the present invention is to provide a process for the preparation of Abametapir.

In one aspect, the present invention is to provide a process for the preparation of abametapir comprising the steps of:
a) forming a reaction mixture of 2-Bromo-5-methylpyridine, ethylene glycol, a base and a catalyst,
b) optionally adding a second solvent,
c) heating the reaction mixture,
d) extracting the reaction mixture,
e) isolating Abametapir.

In another aspect, the present invention is to provide Abametapir hydrochloride, Abametapir maleate, Abametapir oxalate, Abametapir sulfate and Abametapir tartarate salts from Abametapir and their preparation thereof.

In another aspect, the present invention is to provide a process for the preparation of Abametapir hydrochloride salt of formula (II)

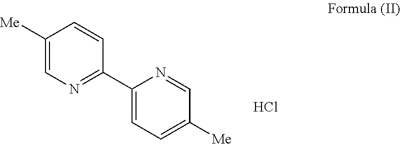 [SMILES: Cc1ccc(-c2ccc(C)cn2)nc1 ; Cl]

comprising the steps of:
a) dissolving Abametapir in an organic solvent,
b) purging Hydrogen chloride,
c) isolating Abametapir hydrochloride salt of formula (II).

In yet another aspect, the present invention is to provide a process for the preparation of Abametapir maleate salt of formula (II)

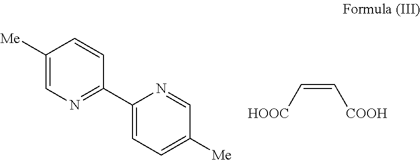 [SMILES: Cc1ccc(-c2ccc(C)cn2)nc1 ; O=C(O)/C=C\C(=O)O]

comprising the steps of:
a) dissolving Abametapir in an organic solvent,
b) adding maleic acid,
c) isolating Abametapir maleate salt of formula (III).

In yet another aspect, the present invention is to provide a process for the preparation of Abametapir oxalate salt of formula (IV)

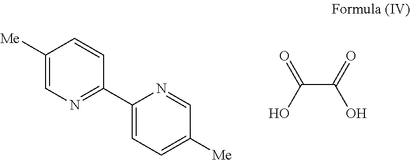 [SMILES: Cc1ccc(-c2ccc(C)cn2)nc1 ; O=C(O)C(=O)O]

comprising the steps of:
a) dissolving Abametapir in an organic solvent,
b) adding oxalic acid,
c) isolating Abametapir oxalate salt of formula (IV).

In yet another aspect, the present invention is to provide a process for the preparation of Abametapir sulfate salt of formula (V)

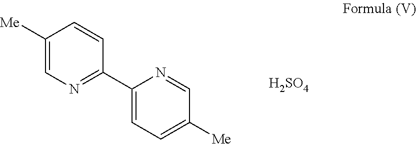 [SMILES: Cc1ccc(-c2ccc(C)cn2)nc1 ; "H2SO4"]

comprising the steps of:
a) dissolving Abametapir in an organic solvent,
b) adding sulfuric acid,
c) isolating Abametapir sulfate salt of formula (V).

In yet another aspect, the present invention is to provide a process for the preparation of Abametapir Tartarate salt of formula (VI)

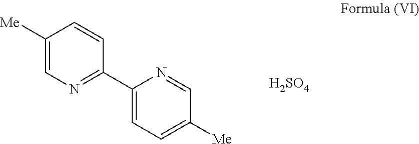 [SMILES: Cc1ccc(-c2ccc(C)cn2)nc1 ; "H2SO4"]

comprising the steps of:
a) dissolving Abametapir in an organic solvent,
b) adding tartaric acid,
c) isolating Abametapir Tartarate salt of formula (VI).

Further aspects of the present invention together with additional features contributing thereto and advantages accruing there from will be apparent from the following description of embodiments which are shown in the accompanying drawing figures wherein:

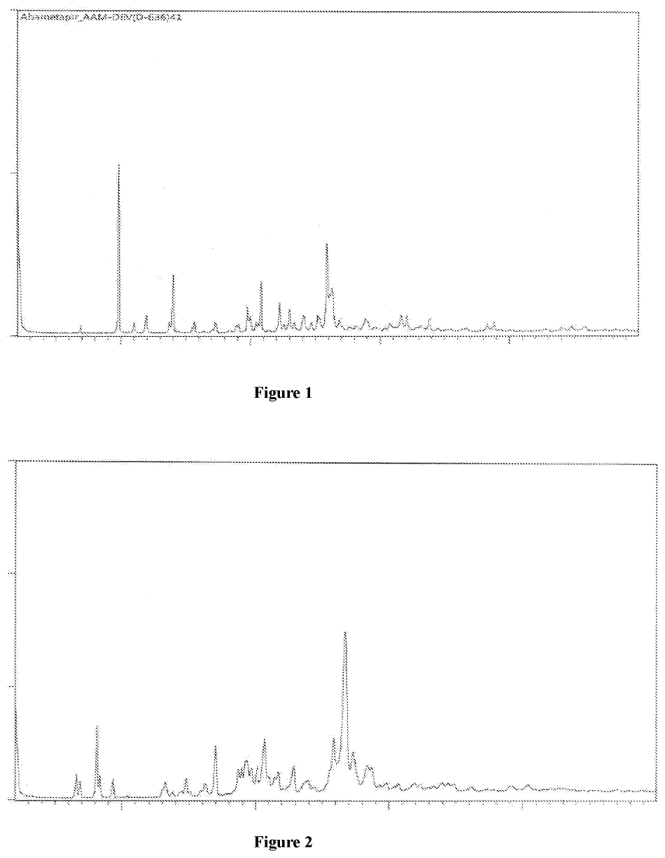

1. FIG. 1 is an X-ray powder diffractogram of Abametapir HCl salt.

2. FIG. 2 is an X-ray powder diffractogram of Abametapir Maleate salt.

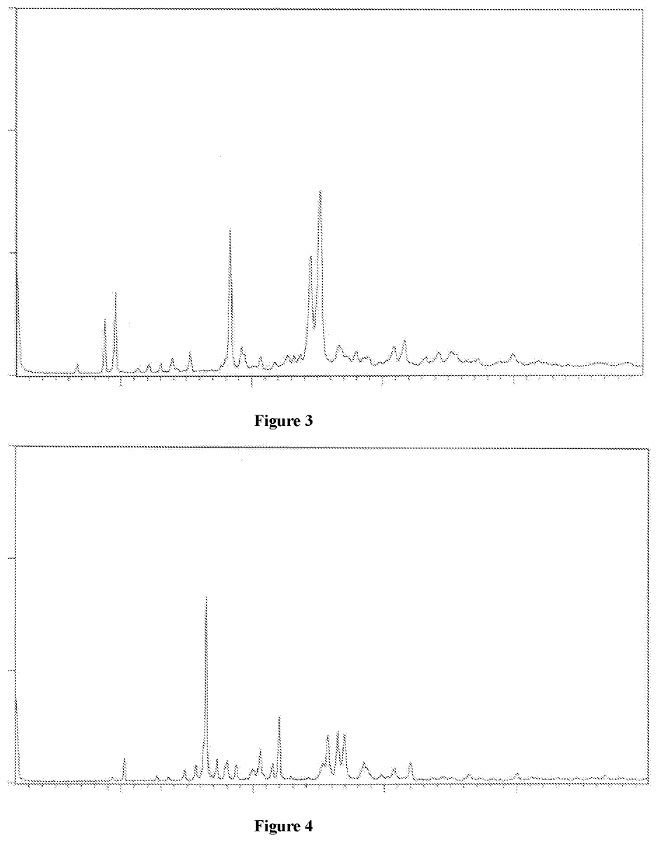

3. FIG. 3 is an X-ray powder diffractogram of Abametapir Oxalate salt.

4. FIG. 4 is an X-ray powder diffractogram of Abametapir Sulfate salt.

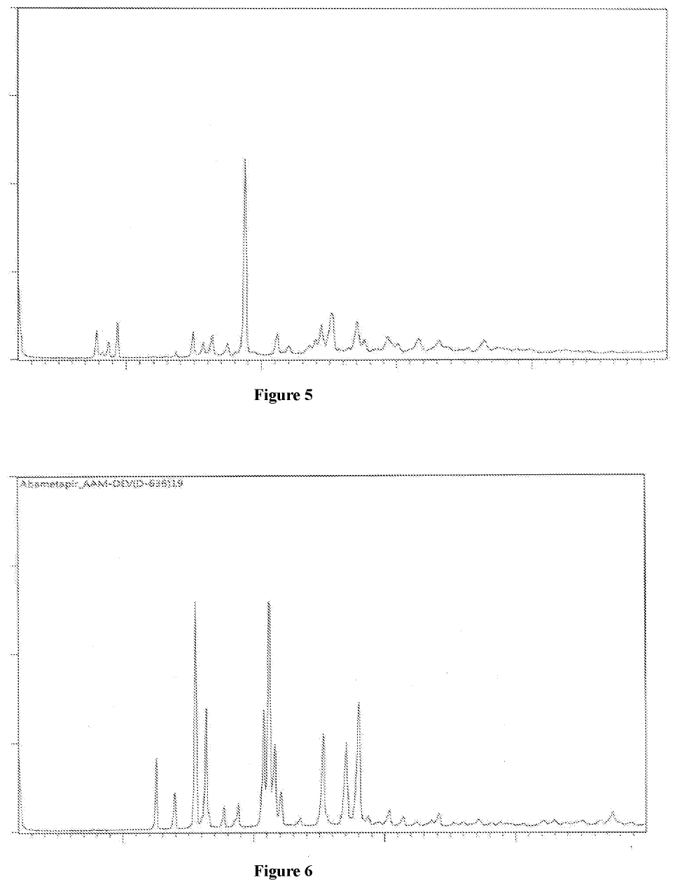

5. FIG. 5 is an X-ray powder diffractogram of Abametapir Tartarate salt.

6. FIG. 6 is an X-ray powder diffractogram of Abametapir free base.

### DETAILED DESCRIPTION OF THE INVENTION

The present invention relates to a process for the preparation of Abametapir and its pharmaceutically acceptable salts.

In one embodiment, the present invention relates to a process for the preparation of abametapir comprising the steps of:
a) forming a reaction mixture of 2-Bromo-5-methylpyridine, ethylene glycol, a base and a catalyst,
b) optionally adding a second solvent,
c) heating the reaction mixture,
d) extracting the reaction mixture,
e) isolating Abametapir.

According to the present invention, a mixture of 2-Bromo-5-methylpyridine, ethylene glycol, an inorganic base selected from sodium hydroxide, potassium hydroxide, sodium carbonate or potassium carbonte, a catalyst such as palladium carbon, optionally a second solvent selected from 1,2-dimethoxy ethane or 1,4-dioxane are heated to about 70 to 110° C. On completion of the reaction, the reaction mixture may be diluted with water and ethyl acetate followed by filteration to remove palladium. The resultant filtrate may be extracted with ethyl acetate and the combined organic layers may be distilled under reduced pressure to obtain abametapir.

According to the present invention, the abametapir obtained above may be recrystallized from aqueous isopropyl alcohol.

The process according to the present invention has advantages over the prior processes which include but not limited to:
1. Lesser reaction time (˜2 h) when compared to use of Palladium acetate process which would approximately takes 65 hours.
2. Heterogenous catalysis, easy and efficient recovery of palladium.
3. Good and improvised yield
4. High productivity

The present invention further relates to Abametapir hydrochloride, Abametapir maleate, Abametapir oxalate, Abametapir sulfate and Abametapir tartarate salts and their preparation thereof.

In one embodiment, the present invention relates to a process for the preparation of Abametapir hydrochloride salt of formula (II)

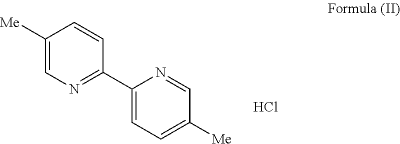 [SMILES: Cc1ccc(-c2ccc(C)cn2)nc1 ; Cl]

comprising the steps of:
a) dissolving Abametapir in an organic solvent
b) purging Hydrogen chloride
c) isolating Abametapir hydrochloride salt of formula (II).

According to the present invention, Abametapir may be dissolved in an organic solvent selected from ester solvents like methyl acetate, ethyl acetate, propyl acetate or butyl acetate, tetrahydrofuran, acetonitrile, dichloromethane, methonal, isopropyl alcohol, Ethonal, methyl ethyl ketone, methyl isobutyl ether, and methyl isopropyl ether and purged with Hydrogen chloride at ambient temperature. The solid obtained may be filtered to obtain Abametapir hydrochloride salt.

In yet another embodiment, Abametapir hydrochloride prepared according to the present invention is characterized by powder X-ray diffraction as depicted in FIG. 1

In yet another embodiment, Abametapir hydrochloride prepared according to the present invention is characterized by powder X-ray diffraction pattern having peaks at 9.82, 14.01, 19.73, 20.79, 22.22, 25.86, 26.30 and 26.83.

In another embodiment, the present invention relates to a process for the preparation of Abametapir maleate salt Formula (III)

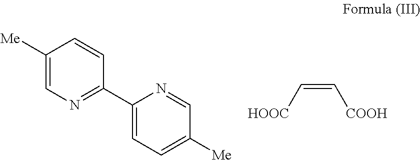 [SMILES: Cc1ccc(-c2ccc(C)cn2)nc1 ; O=C(O)/C=C\C(=O)O]

comprising the steps of:
a) dissolving Abametapir in an organic solvent,
b) adding maleic acid,
c) isolating Abametapir maleate salt of formula (III).

According to the present invention, Abametapir may be dissolved in an organic solvent selected from ester solvents like methyl acetate, ethyl acetate, propyl acetate or Butyl acetate, tetrahydrofuran, acetonitrile, dichloromethane, methonal, isopropyl alcohol, Ethonal, methyl ethyl ketone, methyl isobutyl ether, and methyl isopropyl ether and added maleic acid. The solid obtained may be filtered to obtain Abametapir maleate salt.

In yet another embodiment, Abametapir maleate prepared according to the present invention is characterized by powder X-ray diffraction as depicted in FIG. 2

In yet another embodiment, Abametapir maleate prepared according to the present invention is characterized by powder X-ray diffraction pattern having peaks at 8.10, 9.31, 13.21, 14.77, 18.67, 20.69, 25.87, 26.73 and 28.37.

In another embodiment, the present invention relates to a process for the preparation of Abametapir oxalate salt Formula (IV)

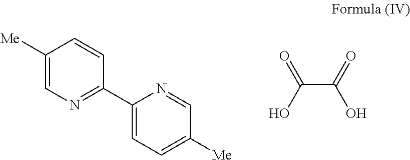 [SMILES: Cc1ccc(-c2ccc(C)cn2)nc1 ; O=C(O)C(=O)O]

comprising the steps of:
a) dissolving Abametapir in an organic solvent,
b) adding oxalic acid,
c) isolating Abametapir oxalate salt of formula (IV).

According to the present invention, Abametapir may be dissolved in an organic solvent selected from ester solvents like methyl acetate, ethyl acetate, propyl acetate or Butyl acetate, tetrahydrofuiran, acetonitrile, dichloromethane, methonal, isopropyl alcohol, Ethonal, methyl ethyl ketone, methyl isobutyl ether, and methyl isopropyl ether and added oxalic acid. The solid obtained may be filtered to obtain Abametapir oxalate salt.

In yet another embodiment, Abametapir oxalate prepared according to the present invention is characterized by powder X-ray diffraction as depicted in FIG. 3.

In yet another embodiment, Abametapir oxalate prepared according to the present invention is characterized by powder X-ray diffraction pattern having peaks at 8.77, 9.58, 18.36, 19.20, 24.52, 25.28 and 31.68.

In another embodiment, the present invention relates to a process for the preparation of Abametapir sulfate salt Formula (V)

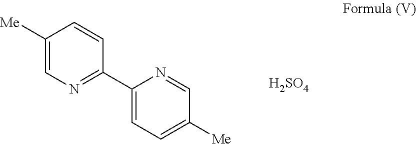 [SMILES: Cc1ccc(-c2ccc(C)cn2)nc1 ; "H2SO4"]

comprising the steps of:
a) dissolving Abametapir in an organic solvent,
b) adding sulfuric acid,
c) isolating Abametapir sulfate salt of formula (V).

According to the present invention, Abametapir may be dissolved in an organic solvent selected from ester solvents like methyl acetate, ethyl acetate, propyl acetate or Butyl acetate, tetrahydrofuran, acetonitrile, dichloromethane, methonal, isopropyl alcohol, Ethonal, methyl ethyl ketone, methyl isobutyl ether, and methyl isopropyl ether and added sulfuric acid. The solid obtained may be filtered to obtain Abametapir sulfate salt.

In yet another embodiment, Abametapir sulfate is is characterized by powder X-ray diffraction as depicted in FIG. 4.

In yet another embodiment, Abametapir sulfate prepared according to the present invention is characterized by powder X-ray diffraction pattern having peaks at 10.23, 16.44, 17.25, 18.02, 20.53, 21.97, 25.70, 26.98 and 28.45.

In another embodiment, the present invention relates to a process for the preparation of Abametapir tartarate salt Formula (VI)

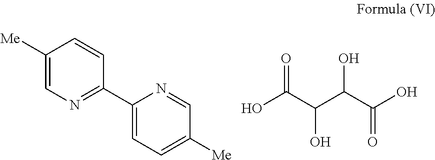 [SMILES: Cc1ccc(-c2ccc(C)cn2)nc1 ; O=C(O)C(O)C(O)C(=O)O]

comprising the steps of:
a) dissolving Abametapir in an organic solvent,
b) adding tartaric acid,
c) isolating Abametapir tartarate salt of formula (VI).

According to the present invention, Abametapir may be dissolved in an organic solvent selected ester solvents like methyl acetate, ethyl acetate, propyl acetate or Butyl acetate, tetrahydrofuran, acetonitrile, dichloromethane, methonal, isopropyl alcohol, Ethonal, methyl ethyl ketone, methyl isobutyl ether, and methyl isopropyl ether and added tartaric acid. The solid obtained may be filtered to obtain Abametapir Tartarate salt.

In yet another embodiment, Abametapir tartarate is is characterized by powder X-ray diffraction as depicted in FIG. 5.

In yet another embodiment, Abametapir Tartarate prepared according to the present invention is characterized by powder X-ray diffraction pattern having peaks at 9.35, 14.92, 18.80, 21.16, 23.51, 25.22, and 26.38.

According to the present invention, Abametapir salts may be isolated by conventional methods such as filtration, solvent removal by distillation under vacuum etc.

In yet another embodiment, Abametapir is characterized by powder X-ray diffraction as depicted in FIG. 6.

In yet another embodiment, Abametapir prepared according to the present invention is characterized by powder X-ray diffraction pattern having peaks at 12.26, 15.26, 16.05, 20.88, 21.30, 25.05 and 27.74.

Alternately, Abametapir used in the present invention is prepared by the process disclosed in Tetrahedron Letters V.039 Year 1998 Pg 2559.

The following examples should not be considered exhaustive, but merely illustrative of only a few of the many aspects and embodiments contemplated by the present disclosure.

### EXAMPLE

### Example 1: Preparation of Abametapir

Under nitrogen atmosphere, a mixture of 2-Bromo-5-methyl pyridine (8 mmol), potassium carbonate (8 mmol), alladium acetate (0.4 mmol) and tetra-n-butlammonium bromide (4 mmol) in N,N-dimethylformamide (2 volumes, based on 2-Bromo-5-methyl pyridine), water (1 volume, 2-Bromo-5-methyl pyridine) and isopropyl alcohol (0.5 volume, based on 2-Bromo-5-methyl pyridine) was heated to 95-100° C. and stirred for 48 hrs. After reaction completion, reaction mass was cooled to room temperature, filtered through hyflo and washed with isopropyl alcohol. Obtained filtrate was concentrated under vacuum and resulted residue was diluted with water, stirred and precipitated solid was filtered. This crude material was further purified by recrystallization in isopropyl alcohol/water to afford Abametapir.

### Example 2: Preparation of Abametapir Hydrochloride

To a solution of Abametapir in ethyl acetate (15 volumes), hydrogen chloride gas was purged and stirred the contents at room temperature. The precipitated solid was filtered, washed with ethyl acetate and dried under vacuum to afford Abametapir Hydrochloride.

### Example 3: Preparation of Abametapir Maleate

To a solution of Abametapir in ethyl acetate (15 volumes), maleic acid (1 eq.) was added and contents were stirred at room temperature. The precipitated solid was filtered, washed with ethyl acetate and dried under vacuum to get Abametapir maleate.

### Example 4: Preparation of Abametapir Oxalate

To a solution of Abametapir in ethyl acetate (15 volumes), oxalic acid (1 eq.) was added and contents were stirred at room temperature. The precipitated solid was filtered, washed with ethyl acetate and dried under vacuum to get Abametapir oxalate.

### Example 5: Preparation of Abametapir Sulfate

To a solution of Abametapir in ethyl acetate (15 volumes), sulfuric acid (1 eq.) was added and contents were stirred at room temperature. The precipitated solid was filtered, washed with ethyl acetate and dried under vacuum to get Abametapir sulfate.

### Example 6: Preparation of Abametapir Tartarate

To a solution of Abametapir in methanol (10 volumes), tartaric acid (0.5 eq.) was added and contents were stirred at room temperature. The precipitated solid was filtered, washed with ethyl acetate and dried under vacuum to get Abametapir tartarate.

### Example 7: Process Description of 2-Bromo-5-methylpyridine

2-Amino-5-methylpyridine (100 g, 0.9247 mol.) and), bromine (295.56 g, 1.8495 mol.) and aqueous sodium nitrite solution (127.61 g, 1.8495 mol., in 300 ml water) were added slowly and sequentially to precooled hydrobromic acid (300 ml, 5±5° C.). Thereafter, stirring was continued at 12±3° C. to complete the reaction. After completion, add 25% w/w aqueous sodium hydroxide solution (800 ml) by maintaining same temperature to precipitate the product. After complete precipitation, product was filtered, washed with precooled water (2×100 ml, <20° C.) and kept under squeezing to yield 120 g (75%) of 2-Bromo-5-methylpyridine as a white solid

### Example 8: Process for the Preparation of Abametapir

Process a: Under inert atmosphere, a mixture of 2-Bromo-5-methylpyridine (100 g, 0.5813 mol.), ethylene glycol (300 ml), 10% w/w palladium on carbon (10 g, 50% w/w, wet) and aqueous sodium hydroxide (46.5 g, 1.1626 mol., in 300 ml water) was heated to 106±3° C. and stirred for 2 hrs. After reaction completion, reaction mass was diluted with water (500 ml) and ethyl acetate (1000 ml) and filtered through hyflo to recover palladium on carbon. From the filtrate, organic layer was separated and aqueous layer was extracted with ethyl acetate (300 ml). Thereafter, combined organic layer was treated with activated carbon at room temperature. After filtration, obtained filtrate was concentrated under reduced pressure. Finally, product was crystallized with aqueous isopropyl alcohol and dried to yield Abametapir as a white solid (42 g, 79%).

Process b: Under inert atmosphere, a mixture of 2-Bromo-5-methylpyridine (100 g, 0.5813 mol.), ethylene glycol (36 g, 0.5813 mol.), 10% w/w palladium on carbon (10 g, 50% w/w, wet) and aqueous sodium hydroxide (46.5 g, 1.1626 mol., in 300 ml water) in 1,2-dimethoxy ethane (500 ml) was heated to 77±3° C. and stirred for 16 hrs. After reaction completion, reaction mass was diluted with water (500 ml) and ethyl acetate (1000 ml) and filtered through hyflo to recover palladium on carbon. From the filtrate, organic layer was separated and aqueous layer was extracted with ethyl acetate (300 ml). Thereafter, combined organic layer was treated with activated carbon at room temperature. After filtration, obtained filtrate was concentrated under reduced pressure. Finally, product was crystallized with aqueous isopropyl alcohol and dried to yield Abametapir as a white solid (37 g, 70%).

Process c: Under inert atmosphere, a mixture of 2-Bromo-5-methylpyridine (100 g, 0.5813 mol.), ethylene glycol (36 g, 0.5813 mol.), 10% w/w palladium on carbon (10 g, 50% w/w, wet) and aqueous sodium hydroxide (46.5 g, 1.1626 mol., in 300 ml water) in 1,4-dioxane (500 ml) was heated to 86±3° C. and stirred for 16 hrs. After reaction completion, reaction mass was diluted with water (500 ml) and ethyl acetate (1000 ml) and filtered through hyflo to recover palladium on carbon. From the filtrate, organic layer was separated and aqueous layer was extracted with ethyl acetate (300 ml). Thereafter, combined organic layer was treated with activated carbon at room temperature. After filtration, obtained filtrate was concentrated under reduced pressure. Finally, product was crystallized with aqueous isopropyl alcohol and dried to yield Abametapir as a white solid (37 g, 70%).

1. A process for the preparation of abametapir comprising the steps of:
a) forming a reaction mixture of 2-Bromo-5-methylpyridine, ethylene glycol, a base and a catalyst,
b) optionally adding a second solvent,
c) heating the reaction mixture,
d) extracting the reaction mixture,
e) isolating abametapir.

2. The process as claimed in claim 1, wherein the catalyst is palladium carbon and the base is selected from the group consisting of sodium hydroxide, potassium hydroxide, sodium carbonate, and potassium carbonate.

3. The process as claimed in claim 1, wherein the second solvent is 1,2-dimethoxy ethane or 1,4-dioxane.

4. The process as claimed in claim 1, wherein the reaction mass is heated to about 70 to 110° C.

5. The process as claimed in claim 1, wherein the reaction mass is diluted with water and ethyl acetate before extraction.

6. The process as claimed in claim 1, wherein extraction is carried by ethyl acetate.

7. The process as claimed in claim 1, wherein the abametapir is recrystallized from aqueous isopropyl alcohol.

8.-32. (canceled)

33. A compound selected from the group consisting of:
abametapir hydrochloride salt,
abametapir maleate salt,
abametapir oxalate salt,
abametapir sulfate salt, and
abametapir tartarate salt.

34. The compound of claim 33 that is abametapir hydrochloride salt and characterized by the powder X-ray diffraction as depicted in FIG. 1, or a powder X-ray diffraction pattern having peaks at 9.82, 14.01, 19.73, 20.79, 22.22, 25.86, 26.30 and 26.83.

35. The compound of claim 33 that is abametapir maleate salt and characterized by the powder X-ray diffraction as depicted in FIG. 2, or a powder X-ray diffraction pattern having peaks at 8.10, 9.31, 13.21, 14.77, 18.67, 20.69, 25.87, 26.73 and 28.37.

36. The compound of claim 33 that is abametapir oxalate salt and characterized by the powder X-ray diffraction as depicted in FIG. 3, or a powder X-ray diffraction pattern having peaks at 8.77, 9.58, 18.36, 19.20, 24.52, 25.28 and 31.68.

37. The compound of claim 33 that is abametapir sulfate salt and characterized by the powder X-ray diffraction as depicted in FIG. 4, or a powder X-ray diffraction pattern having peaks at 10.23, 16.44, 17.25, 18.02, 20.53, 21.97, 25.70, 26.98 and 28.45.

38. The compound of claim 33 that is abametapir tartarate salt and characterized by the powder X-ray diffraction as depicted in FIG. 5, or a powder X-ray diffraction pattern having peaks at 9.35, 14.92, 18.80, 21.16, 23.51, 25.22, and 26.38.

39. A process for the preparation of an abametapir salt comprising the steps of:
when the abametapir salt is abametapir hydrochloride, then
a) dissolving abametapir in an organic solvent,
b) purging hydrogen chloride, and
c) isolating abametapir hydrochloride salt of formula (II); or
when the abametapir salt is abametapir maleate, then
a) dissolving abametapir in an organic solvent,
b) adding maleic acid, and
c) isolating abametapir maleate salt of formula (III); or
when the abametapir salt is abametapir oxalate, then
a) dissolving abametapir in an organic solvent,
b) adding oxalic acid, and
c) isolating abametapir oxalate salt of formula (IV); or
when the abametapir salt is abametapir sulfate, then
a) dissolving abametapir in an organic solvent,
b) adding sulfuric acid, and
c) isolating abametapir sulfate salt of formula (V); or
when the abametapir salt is abametapir tartarate, then
a) dissolving abametapir in an organic solvent,
b) adding tartaric acid, and
c) isolating abametapir tartarate salt of formula (VI).

40. The process of claim 39, wherein the organic solvent is selected from the group consisting of methyl acetate, ethyl acetate, propyl acetate, butyl acetate, tetrahydrofuran, acetonitrile, dichloromethane, methanol, isopropyl alcohol, ethanol, methyl ethyl ketone, methyl isobutyl ether, and methyl isopropyl ether.
